(19) United States
(12) Patent Application Publication
(10) Pub. No.: US 2023/0189814 A1
(43) Pub. Date: Jun. 22, 2023

(54) SEED TREATMENT COMPOSITIONS THAT INCREASE MICROORGANISM LONGEVITY

(71) Applicant: PRO FARM GROUP, INC., Davis, CA (US)

(72) Inventors: Kalle Virta, Vantaa (FI); Virge Vasar, Vantaa (FI); Sergio Pieroni, Vantaa (FI); Pankaj Pathak, Davis, CA (US)

(21) Appl. No.: 17/987,584

(22) Filed: Nov. 15, 2022

Related U.S. Application Data
(60) Provisional application No. 63/244,877, filed on Sep. 16, 2021.

(51) Int. Cl. A01N 63/20; A01N 25/10; A01P 21/00

(52) U.S. Cl. CPC A01N 63/20; A01N 25/10; A01P 21/00

(57) ABSTRACT

The present disclosure includes seed coating compositions and methods for coating seeds that can improve the longevity of microbes that are inoculated on the seeds.

### PRIORITY CLAIM

The present application claims priority to U.S. Provisional Application No. 63/244,877, filed Sep. 16, 2021 and titled SEED TREATMENT COMPOSITIONS THAT INCREASE MICROORGANISM LONGEVITY (“the '877 Provisional application”). The '877 Provisional Application is hereby incorporated herein in its entirety.

### TECHNICAL FIELD

The present disclosure relates in general to the field of seed treatment chemicals.

### BACKGROUND

Without limiting the scope of the disclosure, its background is described in connection with seed coating materials that can prolong the longevity of a microorganism that is coated on the seeds.

A wide range of microorganisms are beneficial to plants in many ways. Beneficial microorganisms can include diverse range of fungi and bacteria as well as nematodes and protozoan organisms. Some microorganisms are beneficial in that they increase the amounts of available nitrogen or phosphorous in the soil surrounding the plants. Others decompose organic materials in the soil, thereby releasing plant nutrients, while other microorganisms assist by regulating the numbers or activities of potentially harmful organisms that may be present in the vicinity of the plants. Still other microorganisms help plants by fixing nitrogen from the atmosphere. For example, some microorganisms can fix nitrogen by combining the nitrogen chemically with other elements to form more reactive nitrogen compounds, such as ammonia, nitrates, or nitrates. This latter group of microorganisms includes bacteria which form a symbiotic relationship with leguminous plants. These bacteria, often referred to as rhizobia, fix atmospheric nitrogen (N₂) when associated with leguminous plants by forming root nodules, thus providing the host plant with most or all of its nitrogen requirements and reducing the need for nitrogen fertilizer. Nitrogen fixation by Rhizobium bacteria is also beneficial because nitrogenous nutrients are left in the soil for non-nitrogen-fixing crops that may be planted later. This beneficial relationship has led to increased research on optimizing the relationship between leguminous plants and rhizobia with the goal of increasing yield.

A degree of specificity exists between the partner species in the N₂-fixing symbiotic relationship. Species of Rhizobium and those in the closely related genera of Mesorhizobium, Sinorhizobium, or Bradyrhizobium all are associated with leguminous plants, such as Mesorhizobium loti on species of Lotus species; Sinorhizobium fredii on Glycine max (soybean), Sinorhizobium meliloti on Medicago species, Melilotus species and Trigonella species; Bradyrhizobium japonicum on Glycine max and Vigna unguiculata (cowpea).

Although rhizobia can survive saprophytically in the soil, many soils lack or contain only low numbers of indigenous rhizobia. Furthermore, these indigenous rhizobia may be only moderately effective in symbiotic nitrogen fixation. In these cases, it is an established practice to apply effective strains of rhizobia as inoculants so as to ensure optimum crop yields.

In addition to rhizobia, other microorganisms may also be applied as beneficial inoculants on seeds. Some of these microorganisms include plant growth promoting microorganisms and biocontrol agents, examples include microorganism strains from the bacterial genera Pseudomonas, Serratia, Bacillus, Azotobacter, Enterobacter, Azospirillum and Burkholderia, as well as Actinomycetes such as Streptomyces, and even cyanobacteria. Fungal genera such as Gliocladium, Trichoderma, Coniothyrium, and Verticillium may also be applied as inoculants (see McQuilken, M P, Halmer P and Rhodes, D. J, 1998, Application of Microorganisms to Seeds; In Formulation of Microorganism Biopesticides: Beneficial microorganisms, nematodes and seed treatments, Ed. H. D. Burges, Kluwer Academic Publishers, pp 255-285; Whipps, J. M. 1997, Developments in the biological control of soil-borne plant pathogens, Advances in Botanical Research 26, 1-134).

However, once coated, the bacteria population declines over time, even under optimum storage conditions. Additionally, there can be issues where local microbe populations can overwhelm and replace the coated microorganisms. This can be problematic because it may mean that low-nitrogen fixing strains replace high-nitrogen fixing strains. Therefore, there is a need for a new seed coating composition and method that can prolong the microorganism (such as rhizobia) longevity on a seed, thereby optimizing crop yield.

In addition, seed coating industry frequently coats seeds with multiple layers of insecticides and/or fungicides alongside with bacteria; these can be problematic because they may interfere with beneficial bacteria that is being coated.

### SUMMARY OF THE DISCLOSURE

This present disclosure provides a seed-coating co-polymer material (CPFAPH) that increases microorganism inoculant lenitively and method for improving the survival and viability of microorganism inoculants on seed. This disclosure also extends to the survival and viability of non-spore forming microorganism inoculants during air storage post-inoculation onto seeds. The disclosure demonstrates the increasing survival and viability of the microorganisms on the stored seed product, thus enabling inoculation well in advance of planting while still preserving the full benefits of the inoculant, seed viability and seed plantability.

In an aspect, the present disclosure relates to prolonging, or maintaining the amount of bacteria coated on seeds in conjunction of insecticide and/or fungicide coatings by having a co-polymer (CPFAPH) material alongside with the insecticide/fungicide coatings.

The present disclosure also relates to at least about the following 15 embodiments:

In an aspect, embodiment 1 relates to a seed coating composition comprising a co-polymer and bacteria, wherein said seed coating composition increases longevity of the bacteria inoculant on a coated seed.

Embodiment 2 is wherein said co-polymer comprises CPFAPH.

Embodiment 3 is wherein said seed in embodiment 1 is a glycine max seed.

Embodiment 4 is wherein said seed in embodiment 1 is a legume.

Embodiment 5 is wherein said legume seed in embodiment 4 is selected from alfalfa, chickpea, and bean.

Embodiment 6 is wherein said microorganism of embodiment 1 is a member of the genus Rhizobium.

Embodiment 7 is wherein said microorganism of embodiment 1 is a plant growth promoting rhizobacteria.

Embodiment 8 relates to a method to increase microorganism longevity on a seed comprising the step of coating said seed with the seed coating composition of embodiment 2, in an amount effective to increase said microorganism longevity.

Embodiment 9 is wherein said microorganism in embodiment 8 is a member of the genus Rhizobium.

Embodiment 10 is wherein said microorganism in embodiment 8 is a plant growth promoting rhizobacteria.

Embodiment 11 is wherein said seed in embodiment 8 is a glycine max seed.

Embodiment 12 is wherein said seed in embodiment 8 is a legume.

Embodiment 13 is wherein said legume seed in embodiment 12 is selected from alfalfa, chickpea, and bean.

Embodiment 14 relates to a method to improve plant yield comprising the step of coating the seed of said plant with a seed coating composition comprising CPFAPH before coating the seed with a plant growth promoting rhizobacteria.

Embodiment 15 is wherein embodiment 14 further comprising the step of adding a pesticide, insecticide and/or a fungicide.

### BRIEF DESCRIPTION OF THE DRAWINGS

For a more complete understanding of the features and advantages of the present disclosure, reference is now made to the detailed description of the disclosure along with the accompanying figures and in which:

FIG. 1 denotes root, shoot and nodule development of alfalfa with and without UBP coating.

FIG. 2 denotes the CFU count for each sample over time. The temperature was initially held at 10° C.

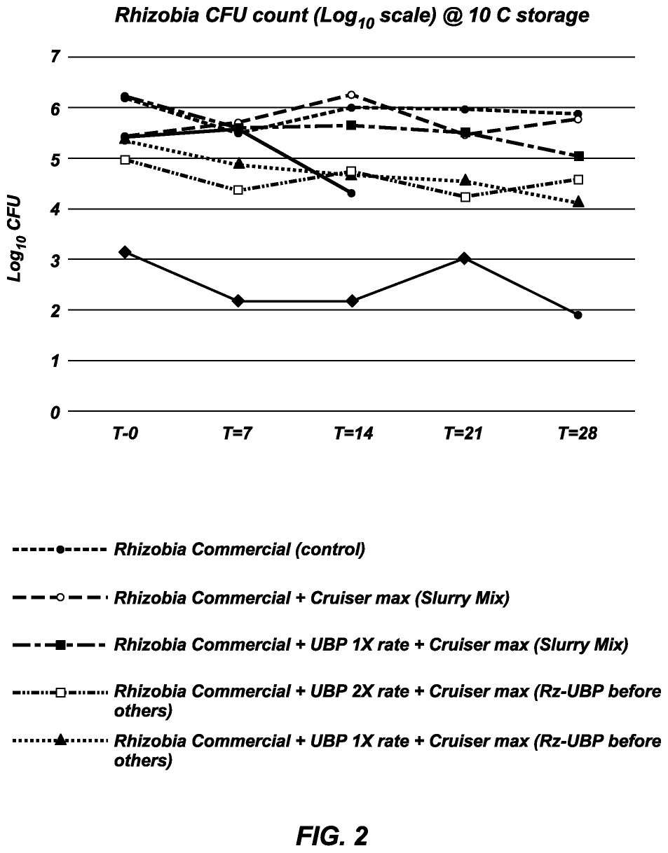

FIG. 3 denotes the same conditions as FIG. 2, but the temperatures were later increased to 30° C. to show the stabilizing effects of co-polymer under different storage conditions.

### DETAILED DESCRIPTION

While the making and using of various embodiments of the present disclosure are discussed in detail below, it should be appreciated that the present disclosure provides many applicable inventive concepts that can be embodied in a wide variety of specific contexts. The specific embodiments discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill the areas relevant to the present disclosure. Terms such as “a”, “an” and “the” are not intended to refer to only a singular entity but include the general class of which a specific example may be used for illustration. The terminology herein is used to describe specific embodiments of the disclosure, but their usage does not delimit the disclosure, except as outlined in the claims.

As used herein, the term “CPFAPH” refers to a co-polymer of fulvic acid and poly-metallic humates. The detailed structure and method of making can be found in U.S. Pat. Nos. 9,738,567 or 10,407,354, or PCT application publication number WO/2019/040460. All of which are incorporated herein by reference in their entirety. Briefly, the structure includes a chemical formula of, for example, (C₁₄H₁₂O₈)ₘ [C₉H₈(K;Na;Mg)₂O₄]n and a schematic structural formula of, for example, FA-(K)-HA, FA-(K;Na)-HA, FA-(K;Na;Mg)-HA, etc., where FA is fulvic acid and HA is humic acid.

As used herein, “UBP” stands for Universal Bio Protector. UBP is a humate based fertilizer which is readily water-soluble and adapted for seed treatment and foliar application. UBP generally includes a humic compound (“CPFAPH”), chelated micronutrients, and biologically active metallic catalysts. UBP has a structure like humic and fulvic acids with 15-16 macro and micronutrients. Humic and fulvic acids are known for keeping more water in their structure and might be used as food by microorganisms. It is also known that UBP has the possibility of acting as a biopolymer for binding purposes. UBP is a natural product derived from cellulose production. The lignosulfonates obtained as a by-product of this process is reproduced to obtain the “supramolecule” of UBP. Methods of producing UBP are presented in U.S. Pat. No. 10,407,354, which is incorporated in reference in its entirety.

As used herein, any term of degree such as, but not limited to, “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

As used herein, the term “chelate” refers to a compound containing a ligand bonded to a central metal atom at two or more points.

As used herein, the terms “effective amount”, “effective concentration”, or “effective dosage” means the amount, concentration, or dosage of the one or more microbially stabilizing compounds sufficient to support microbial activity on one or more seeds. The actual effective dosage in absolute value depends on factors including, but not limited to, the amount (e.g., the concentration, volume, of compound, etc.) of microbes to be stabilized, and the stability of the one or more microbially stabilizing compounds in compositions and/or as plant or plant part treatments. The “effective amount”, “effective concentration”, or “effective dosage” of the one or more microbially stabilizing compounds may be determined, e.g., by a dose response experiment.

As used herein, the term “carrier” means an “agronomically acceptable carrier.” An “agronomically acceptable carrier” means any material which can be used to deliver the actives (e.g., microbially stabilizing compounds described herein, agriculturally beneficial ingredient(s), biologically active ingredient(s), etc.) to a plant or a plant part (e.g., a seed), and preferably which carrier can be applied (to the plant, plant part (e.g., a seed), or soil) without having an adverse effect on plant growth, soil structure, soil drainage or the like.

As used herein, term “enhanced plant growth” means increased plant yield (e.g., increased biomass, increased fruit number, increased boll number, or a combination thereof that may be measured by bushels per acre), increased root number, increased root mass, increased root volume, increased leaf area, increased plant stand, increased plant vigor, faster seedling emergence (i.e., enhanced emergence), faster germination, (i.e., enhanced germination), or combinations thereof.

As used herein, the term “inoculum” means any form of microbial cells, or spores, which is capable of propagating on or in the soil when the conditions of temperature, moisture, etc., are favorable for microbial growth.

As used herein, “CFU” means Colony Forming Unit. CFU is used as a measurement of the number of viable bacteria cells in a sample.

As used herein, “increasing microorganism longevity” means increasing the survival rate of at least one microorganism, such as rhizobia, on a seed. Increases to microorganism longevity can be measured by the increase number of colonies forming units (CFU) per seed over time.

Legumes have a symbiotic relationship with Rhizobium bacteria. The bacteria inhabit the nodules of legume roots and convert, or “fix,” atmospheric nitrogen into biologically useful nitrogen for synthesis of essential biological compounds, such as proteins. Nitrogen fixation by Rhizobium bacteria is also beneficial because nitrogenous nutrients are left in the soil for non-nitrogen-fixing crops that may be planted later. Peas, beans and alfalfa are examples of agriculturally important legumes that are capable of symbiotic relationships with Rhizobium bacteria. Other legumes can include clover, chickpeas, lentils, lupins, mesquite, carob, soybeans, peanuts, and tamarind. It is envisioned that this symbiotic relationship may apply to all legumes. Further other plants exhibit comparable symbiotic relationships with microbes.

Each different Rhizobium species is capable of forming symbiotic relationships with only a specific legume species. For example, Rhizobium japonicum only nodulates the roots of soybeans. Common agricultural practice is to inoculate either the soil or the legume seeds themselves with an appropriate Rhizobium culture. This inoculation encourages efficient symbiosis, which leads to enhanced legume crop yield.

Because of the need of increasing legume crop yield, a great deal of research has gone into improving the nitrogen-fixing ability of Rhizobium strains. Unfortunately, while improved Rhizobium strains may exhibit enhanced nitrogen-fixing capacity in laboratory or greenhouse trials, the improved strains often die off or are outcompeted by indigenous Rhizobium strains which may have lesser nitrogen-fixing abilities. Thus introduced, high nitrogen-fixing Rhizobium strains may be ineffective in increasing crop yield under field conditions because the indigenous, low nitrogen-fixing strains—have evolved to grow more efficiently in that particular soil or environmental condition and may overwhelm the introduced strain, unless the number of inoculants are increased, typically above about 1,000 CFU per seed at initial inoculation.

In addition, seeds of leguminous plants, like any other agricultural crop, may need to be treated with fungicides and/or insecticides before planting. These chemicals applied as seed dressings protect germinating seeds and young seedlings against various fungal pathogens and pests. However, when rhizobial inoculants are introduced onto chemically treated seeds of a legume crop, survival of the bacteria on such seeds can be markedly reduced due to possible toxic effects of pesticides. Therefore, in one embodiment, this disclosure relates to a method for preserving and stabilizing bacteria and other microorganisms on one or more seeds in combination with chemical substances. A non-limiting example include using CPFAPH which has fulvic acid and poly-metallic humates coatings with adherents plus Rhizobium bacteria to seed coat alfalfa seeds.

There are three basic forms of commercial inoculant: solid, liquid and freeze-dried. The most commonly used are solid, peat-based inoculants that can be purchased for seed or direct soil application. Liquid inoculants are available in broth culture or as frozen concentrate. Broth or frozen concentrates usually are mixed with water and sprayed into the seed furrow at planting. Because liquid inoculants must be kept frozen or refrigerated during shipment and storage, their availability through normal distribution channels is limited.

Seed-applied inoculants exist as planter box additives, pre-inoculated seed and custom inoculants. The planter box additive, where inoculant is mixed with seed in the planter box, is most common. This can be accomplished by applying dry inoculum or a slurry directly to the seed. The dry method is least desirable because of uneven distribution and poor adhesion of inoculum to seed. The slurry is prepared by mixing the inoculum with water for better adherence to the seed coat. Seed also may be prewetted before mixing it with dry inoculants. One typically cannot leave dry inoculum in the planter box overnight, or let it get wet from rain or dew.

Many small-seeded legumes, such as alfalfa, are pre-inoculated by seed conditioners, distributors and dealers. After conditioning, they apply a sticking agent to the seed, followed by the dry inoculum, or they incorporate the inoculum into a seed coating. One typically keeps pre-inoculated seed in a cool environment during shipping and storage, and use the seed within one year of inoculation, or pre-inoculate it prior to planting. Rhizobia cells are living bacteria that must be kept viable until planting.

Custom microbe inoculation usually is done on the farm or by the seed distributor. This involves application of a nutrient-rich adhesive formulation, followed by a peat-based inoculum. This method assures viable inoculum if seed is stored properly following application.

Inoculum contains living rhizobial cells that survive on an organic carrier such as peat. The rhizobia population declines over time, even under proper storage conditions. Most inoculum manufacturers put an expiration date on the package. Outdated inoculum is not desirable because the rhizobial population may have declined significantly after the expiration date.

Optimum storage conditions for peat-base inoculum is under refrigeration at 10 degrees Celsius. It is best not to freeze peat-based inoculant. Ultraviolet rays and heat can kill the bacteria.

The present disclosure keeps in mind the challenges of maintaining a microbial rich inoculum under even optimal storage conditions. Failure to maintain a high CFU will functionally render the bacteria inactive. Therefore, in one embodiment, the present disclosure checks the seed inoculant CFU right after the coating. In addition, the storage conditions are kept constant as time passes in Example 1.

Further, the present disclosure's plants and microorganisms are not limited to legume plants or nitrogen fixing bacteria. The technology can incorporate any plant or microbe. For example, some bacteria associated with the roots of crop plants have beneficial effects on their host and are referred to as plant growth promoting rhizobacteria (PGPR) can also be used to coat seeds. The rhizosphere is subjected to dramatic changes and its dynamic nature creates interactions that result in biocontrol of diseases. PGPR are free living bacteria that may have beneficial effects on plants, viz. seedling emergence, colonizing roots, stimulating overall plant growth, mineral nutrition, and water utilization as well as disease suppression. The presence of rhizobia in the rhizosphere may also protect host roots from damage caused by pathogens. Thus, any known PGPR and any type of plants can be used and is contemplated by this disclosure.

In certain embodiments of the present disclosure, the co-polymer UBP is applied on the seed first, before any microorganism. In certain embodiments, the microorganism inoculants are added to the seeds before the application of UBP. Examples of inoculants that can be used in this disclosure include bacteria like rhizobia. In other embodiments, the inoculants are other bacteria, or fungi or other microorganisms that may be beneficial to plants.

In an embodiment, the beneficial microorganisms can be in a spore form, a vegetative form, or any combination thereof. The one or more beneficial microorganisms can include any number of microorganisms having one or more beneficial properties including, but not limited to, producing one or more of the plant signal molecules described herein, enhancing nutrient and water uptake, promoting and/or enhancing nitrogen fixation, growth, seed germination, or seedling emergence, breaking the dormancy or quiescence of a plant, or providing anti-fungal activity.

In one embodiment, the one or more beneficial microorganisms are diazotrophs (i.e., bacteria which are symbiotic nitrogen-fixing bacteria). In still another embodiment, the one or more beneficial microorganisms are bacterial diazotrophs selected from the genera Rhizobium spp., Bradyrhizobium spp., Azorhizobium spp., Sinorhizobium spp., Mesorhizobium spp., Azospirillum spp., and combinations thereof. In still another embodiment, the one or more beneficial microorganisms are bacteria selected from the group consisting of Rhizobium cellulosilyticum, Rhizobium daejeonense, Rhizobium etli, Rhizobium galegae, Rhizobium gallicum, Rhizobium giardinii, Rhizobium hainanense, Rhizobium huautlense, Rhizobium indigoferae, Rhizobium leguminosarum, Rhizobium loessense, Rhizobium lupini, Rhizobium lusitanum, Rhizobium meliloti, Rhizobium mongolense, Rhizobium miluonense, Rhizobium sullae, Rhizobium tropici, Rhizobium undicola, Rhizobium yanglingense, Bradyrhizobium bete, Bradyrhizobium canadense, Bradyrhizobium elkanii, Bradyrhizobium iriomotense, Bradyrhizobium japonicum, Bradyrhizobium jicamae, Bradyrhizobium liaoningense, Bradyrhizobium pachyrhizi, Bradyrhizobium yuanmingense, Azorhizobium caulinodans, Azorhizobium doebereinerae, Sinorhizobium abri, Sinorhizobium adhaerens, Sinorhizobium americanum, Sinorhizobium aborts, Sinorhizobium fredii, Sinorhizobium indiaense, Sinorhizobium kostiense, Sinorhizobium kummerowiae, Sinorhizobium medicae, Sinorhizobium meliloti, Sinorhizobium mexicanus, Sinorhizobium morelense, Sinorhizobium saheli, Sinorhizobium terangae, Sinorhizobium xinjiangense, Mesorhizobium albiziae, Mesorhizobium amorphae, Mesorhizobium chacoense, Mesorhizobium ciceri, Mesorhizobium huakuii, Mesorhizobium loti, Mesorhizobium mediterraneum, Mesorhizobium pluifarium, Mesorhizobium septentrionale, Mesorhizobium temperatum, Mesorhizobium tianshanense, Azospirillum amazonense, Azospirillum brasilense, Azospirillum canadense, Azospirillum doebereinerae, Azospirillum formosense, Azospirillum halopraeferans, Azospirillum irakense, Azospirillum largimobile, Azospirillum lipoferum, Azospirillum melinis, Azospirillum oryzae, Azospirillum picis, Azospirillum rugosum, Azospirillum thiophilum, Azospirillum zeae, and combinations thereof.

In a particular embodiment, the beneficial microorganism is a bacterial daizotroph selected from the group consisting of Bradyrhizobium japonicum, Rhizobium leguminosarum, Rhizobium meliloti, Sinorhizobium meliloti, Azospirillum brasilense, and combinations thereof. In another embodiment, the beneficial microorganism is the bacterial daizotroph Bradyrhizobium japonicum. In another embodiment, the beneficial microorganism is the bacterial daizotroph Rhizobium leguminosarum. In another embodiment, the beneficial microorganism is the bacterial daizotroph Rhizobium meliloti. In another embodiment, the beneficial microorganism is the bacterial daizotroph Sinorhizobium meliloti. In another embodiment, the beneficial microorganism is the bacterial daizotroph Azospirillum brasilense.

In a particular embodiment, the one or more diazotrophs comprises one or more strains of Rhizobium leguminosarum. In another particular embodiment, the strain of R. leguminosarum comprises the strain S012A-2-(IDAC 080305-01). In another particular embodiment, the one or more diazotrophs comprises a strain of Bradyrhizobium japonicum. In still another particular embodiment, the strain of Bradyrhizobium japonicum comprises the strains B. japonicum USDA 532C, B. japonicum USDA 1 10, B. japonicum USDA 123, B. japonicum USDA 127, B. japonicum USDA 129, B. japonicum NRRL B-50608, B. japonicum NRRL B-50609, B. japonicum NRRL B-50610, B. japonicum NRRL B-5061 1, B. japonicum NRRL B-50612, B. japonicum NRRL B-50592 (deposited also as NRRL B-59571), B. japonicum NRRL B-50593 (deposited also as NRRL B-59572), B. japonicum NRRL B-50586 (deposited also as NRRL B-59565), B. japonicum NRRL B-50588 (deposited also as NRRL B-59567), B. japonicum NRRL B-50587 (deposited also as NRRL B-59566), B. japonicum NRRL B-50589 (deposited also as NRRL B-59568), B. japonicum NRRL B-50591 (deposited also as NRRL B-59570), B. japonicum NRRL B-50590 (deposited also as NRRL B-59569), B. japonicum NRRL B-50594 (deposited also as NRRL B-50493), B. japonicum NRRL B-50726, B. japonicum NRRL B-50727, B. japonicum NRRL B-50728, B. japonicum NRRL B-50729, B. japonicum NRRL B-50730, and combinations thereof.

In still yet a more particular embodiment, the one or more diazotrophs comprises one or more strains of R. leguminosarum comprises the strain 5012A-2-(I DAC 080305-01), B. japonicum USDA 532C, B. japonicum USDA 1 10, B. japonicum USDA 123, B. japonicum USDA 127, B. japonicum USDA 129, B. japonicum NRRL B-50608, B. japonicum NRRL B-50609, B. japonicum NRRL B-50610, B. japonicum NRRL B-5061 1, B. japonicum NRRL B-50612, B. japonicum NRRL B-50592 (deposited also as NRRL B-59571), B. japonicum NRRL B-50593 (deposited also as NRRL B-59572), B. japonicum NRRL B-50586 (deposited also as NRRL B-59565), B. japonicum NRRL B-50588 (deposited also as NRRL B-59567), B. japonicum NRRL B-50587 (deposited also as NRRL B-59566), B. japonicum NRRL B-50589 (deposited also as NRRL B-59568), B. japonicum NRRL B-50591 (deposited also as NRRL B-59570), B. japonicum NRRL B-50590 (deposited also as NRRL B-59569), B. japonicum NRRL B-50594 (deposited also as NRRL B-50493), B. japonicum NRRL B-50726, B. japonicum NRRL B-50727, B. japonicum NRRL B-50728, B. japonicum NRRL B-50729, B. japonicum NRRL B-50730, and combinations thereof.

In another embodiment, the one or more beneficial microorganisms comprise one or more phosphate solubilizing microorganisms. Phosphate solubilizing microorganisms include fungal and bacterial strains. In an embodiment, the phosphate solubilizing microorganism are microorganisms selected from the genera consisting of Acinetobacter spp., Arthrobacter spp, Arthrobotrys spp., Aspergillus spp., Azospirillum spp., Bacillus spp., Burkholderia spp., Candida spp., Chryseomonas spp., Enterobacter spp., Eupenicillium spp., Exiguobacterium spp., Klebsiella spp., Kluyvera spp., Microbacterium spp., Mucor spp., Paecilomyces spp., Paenibacillus spp., Penicillium spp., Pseudomonas spp., Serratia spp., Stenotrophomonas spp., Streptomyces spp., Streptosporangium spp., Swaminathania spp., Thiobacillus spp., Torulospora spp., Vibrio spp., Xanthobacter spp., Xanthomonas spp., and combinations thereof.

In still yet another embodiment, the phosphate solubilizing microorganism is a microorganism selected from the group consisting of Acinetobacter calcoaceticus, Arthrobotrys oligospora, Aspergillus niger, Azospirillum amazonense, Azospirillum brasilense, Azospirillum canadense, Azospirillum doebereinerae, Azospirillum formosense, Azospirillum halopraeferans, Azospirillum irakense, Azospirillum largimobile, Azospirillum lipoferum, Azospirillum melinis, Azospirillum oryzae, Azospirillum picis, Azospirillum rugosum, Azospirillum thiophilum Azospirillum zeae, Bacillus amyloliquefaciens, Bacillus atrophaeus, Bacillus circulans, Bacillus licheniformis, Bacillus subtilis, Burkholderia cepacia, Burkholderia vietnamiensis, Candida krissii, Chryseomonas luteola, Enterobacter aerogenes, Enterobacter asburiae, Enterobacter taylorae, Eupenicillium parvum, Kluyvera cryocrescens, Mucor ramosissimus, Paecilomyces hepialid, Paecilomyces marquandii, Paenibacillus macerans, Paenibacillus mucilaginosus, Penicillium bilaiae (formerly known as Penicillium bilaii), Penicillium albidum, Penicillium aurantiogriseum, Penicillium chrysogenum, Penicillium citreonigrum, Penicillium citrinum, Penicillium digitatum, Penicillium frequentas, Penicillium fuscum, Penicillium gaestrivorus, Penicillium glabrum, Penicillium griseofulvum, Penicillium implicatum, Penicillium janthinellum, Penicillium lilacinum, Penicillium minioluteum, Penicillium montanense, Penicillium nigricans, Penicillium oxalicum, Penicillium pinetorum, Penicillium pinophilum, Penicillium purpurogenum, Penicillium radicans, Penicillium radicum, Penicillium raistrickii, Penicillium rugulosum, Penicillium simplicissimum, Penicillium solitum, Penicillium variabile, Penicillium velutinum, Penicillium viridicatum, Penicillium glaucum, Penicillium fussiporus, Penicillium expansum, Pseudomonas corrugate, Pseudomonas fluorescens, Pseudomonas lutea, Pseudomonas poae, Pseudomonas putida, Pseudomonas stutzeri, Pseudomonas trivialis, Serratia marcescens, Stenotrophomonas maltophilia, Swaminathania salitolerans, Thiobacillus ferrooxidans, Torulospora globosa, Vibrio proteolyticus, Xanthobacter agilis, Xanthomonas campestris, and combinations thereof.

Further, other organic acid co-polymers are contemplated in the present disclosure. In another embodiment, the seeds are glycine max seeds.

### EXAMPLES

### Example 1

The trial was conducted using alfalfa seeds. Seeds treated with UBP and inoculated with a Rhizobacteria were compared with commercially available seeds that were inoculated with Rhizobacteria (without treatment of UBP). The volume of UBP applied as well as the layering of the treatments were varied.

One of the purposes of this experiment was to determine if the addition of UBP to inoculated seeds increased the longevity of the inoculated bacteria on the seed. The amount of an inoculated bacteria on a seed can be measured in terms of CFU. CFU measurements were taken on Day 0, 7, and 14. Longevity of the bacteria is seen by looking at the decrease of bacteria on the seed over this time period. A relatively smaller percentage drop in CFU over a 14 days period would signal an increase in longevity and storage time of the seeds.

Treatment 1—control: seed treatment normally used by commercial companies claiming a minimum of 1000 CFU/seed over 30 days.

Treatment 2: Replace the seed treatment coating with UBP, and in the same amount/ton normally used by the company that is providing the inoculant.

Treatment 3: Replace with UBP using 800 cm3/ton of seed.

Treatment 4: UBP at 800 cm3/ton of seed used in the first layer around the seed, keeping the rest of the components of the seed treatment constant.

Treatment 5: UBP at 400 cm3/ton of seed around the seed and 400 cm3 of UBP with the inoculant.

Results are shown in Table 1 below with CFU (of Rhizobacteria)/seed at T0, T7 (7 days after inoculation) and T14 (14 days after inoculation):

TABLE 1

| CFU/seed | T = 0 | T = 7 days | T-14 days |
|---|---|---|---|
| Treatment 1 | 867 (b) | 666 | 267 |
| Treatment 2 | 467 (b) | 167 | 67 |
| Treatment 3 | 633 (b) | 267 | 100 |
| Treatment 4 | 2000 (a) | 1333 | 400 |
| Treatment 5 | 867 (b) | 367 | 0 |

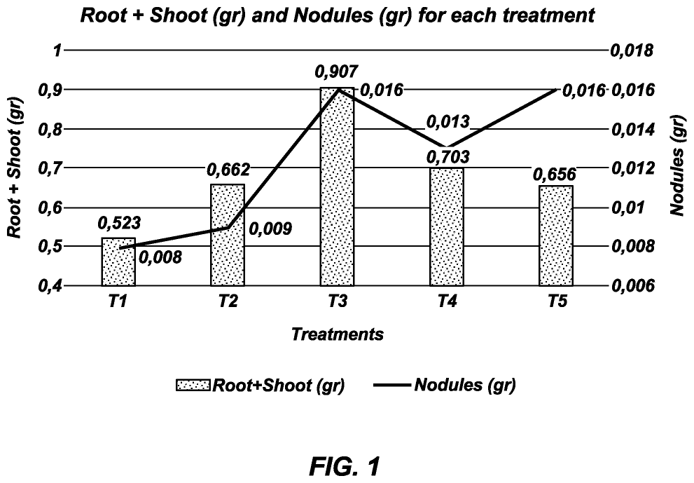

These treated seeds were later planted. The root, shoot and nodule development of alfalfa were also observed as shown in FIG. 1.

UBP is having a positive effect on bacteria survival in absolute number of CFUs, especially in treatment 4 as compare to the control in treatment 1. In addition, UBP is also having positive effects on root/shoot and nodule formation.

### Example 2

Seed treatments were applied to seeds using commercial application rates for the products listed below. Samples were analyzed via microbiology techniques for CFU counts on Rhizobium in a two steps approach:

Step 1: samples were analyzed immediately after seed treatment, and again at 7 days, 15, 21, 30 at 10° C. storage temperature.

Step 2: The samples are then shifted to 30° C. storage and analyzed again for Rhizobia count for T=0, 7, 14 days.

The two-steps approach is applied to address the following 1.) Providing time for biological (Rhizobia) to stabilize on seed after seed treatment 2.) at lower temperature storage (10° C.), measuring the impact of insecticides or fungicides or copolymer on Rhizobia and 3.) Effect of temperature on Rhizobia alone and in presence of chemicals and impact of presence of copolymer in two different concentrations in extending the longevity of Rhizobia.

Test Times: 0, 7, 15, 21, 30 days at 10° C. storage and 0, 7, 14 days at 30° C. storage

Inoculant rates: Rizoliq LLI 250 ml/100 kg of seed (0.43 fl oz/cwt)+Premax: 50 ml/100 kg of seed (0.85 fl oz/cwt). Cwt is equal to 100 lbs of weight. 1 fl oz is 30 ml.

UBP 1× rate: 80 ml/100 kg of seed. (1.37 floz/cwt)

UBP High rate: 120 ml/100 kg of seed (2.06 floz/cwt)

Cruiser Max® and Acceleron® rate: these were applied on seeds with commercially known rate per their label instructions.

TABLE 2
study design

Rhizobia only (control Rhizobia liq + premax)
Rhizobia + Cruiser max (Slurry Mix)
Rhizobia + UBP 1X rate + Cruiser max (Slurry Mix)
Rhizobia + UBP high rate + Cruiser max (Rhizobia + UBP before others)
Rhizobia + UBP 1X rate + Cruiser max (Rhizobia + UBP before others)
Rhizobia + Acceleron (Slurry Mix)

TABLE 3
Commercial control products and the actives listed

| Commercial | | | | |
|---|---|---|---|---|
| Seed | | | | |
| treatment | | | | |
| Product | Insecticide | Fungicide | Fungicide | Fungicide |
| Cruiser max | Cruiser | Apron Max | Maxim 4FS | |
| | 5FS(Thiamethoxan) | (Mefenoxam) | (Fludioxonil) | |
| | @1.28 floz/cwt | @0.32 floz/cwt | @0.08 floz/cwt | |
| Acceleron | Acceleron IX | Aceeleron DC | Acceleron DX | Acceleron DX |
| soybeans | (Imidacloprid) | (Metalaxyl) | (Fluxapyroxad) | (Pyraclostrin) |
| | @3.2 floz/cwt | @0.375 floz/cwt | @0.24 floz/cwt | @0.6 floz/cwt |

Results: 1.) Rhizobia counts stabilized after seed treatment procedure (from T=0 to T=21). 2.) The chemicals (insecticide and fungicides) alone or in combination with copolymer (UBP) did not have a detrimental effect on Rhizobia count. The Rhizobia counts for the two controls had different starting CFU counts, but both stabilized to T-0 levels at the end of 21 days.

TABLE 4
CFU counts over time at 10° C.

| Treatment list and procedure | T = 0 | T = 7 days | T = 14 days | T = 21 days |
|---|---|---|---|---|
| Rhizobia (control Rhizobia liq + premax) | 1.55E+06 | 3.10E+05 | 1.00E+06 | 9.25E+05 |
| Rhizobia + Cruiser max (Slurry Mix) | 2.70E+05 | 5.05E+05 | 1.80E+06 | 2.90E+05 |
| Rhizobia + UBP 1X rate + Cruiser max | 1.68E+06 | 3.95E+05 | 4.45E+05 | 3.20E+05 |
| (Slurry Mix) | | | | |
| Rhizobia + UBP high rate + Cruiser max | 9.35E+04 | 2.30E+04 | 5.45E+04 | 1.74E+04 |
| (Rhizobia + UBP before others) | | | | |
| Rhizobia + UBP 1X rate + Cruiser max | 2.20E+05 | 7.30E+04 | 4.60E+04 | 3.50E+04 |
| (Rhizobia + UBP before others) | | | | |
| Rhizobia + Acceleron (Slurry Mix) | 1.40E+03 | 1.50E+02 | 1.50E+02 | 1.05E+03 |

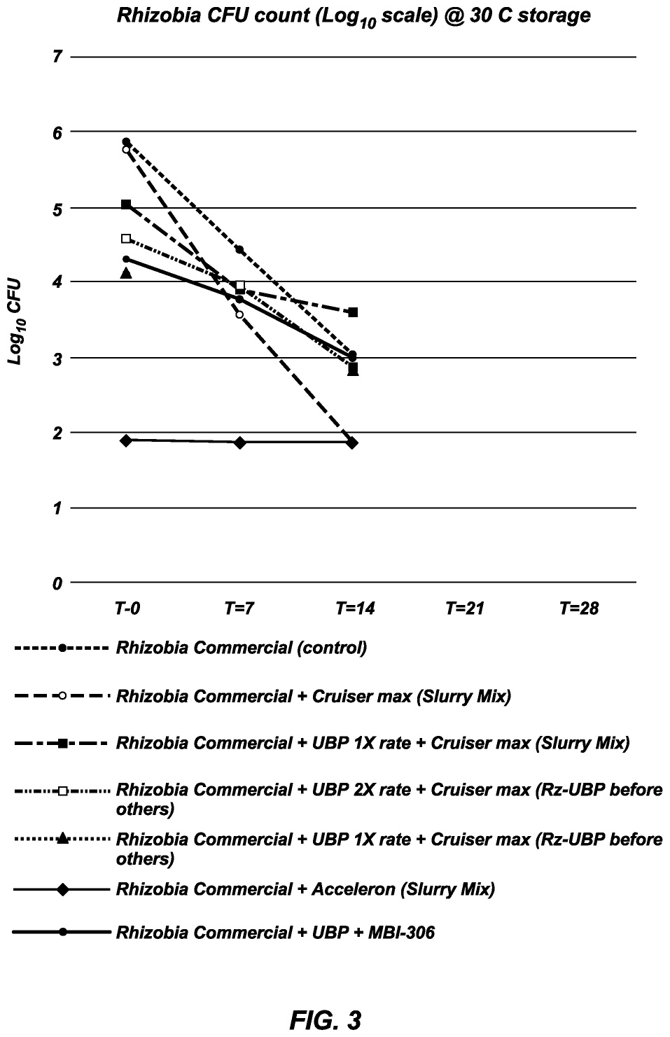

FIG. 3 denotes the same treatment list as Table 4 above; however, the seeds were stored at 30 degrees Celsius and CFU were counted at t=0, 7, and 14 days. The slope of the decrease in microbes can be calculated by one skilled in the art and the lines are visible in FIG. 3. The embodiments with UBP appear to have less sharp slope drop than the control; thereby showing that the microbe longevity is maintained.

It is contemplated that any embodiment discussed in this specification can be implemented with respect to any method, kit, reagent, or composition of the disclosure, and vice versa. Furthermore, compositions of the disclosure can be used to achieve methods of the disclosure.

It will be understood that particular embodiments described herein are shown by way of illustration and not as limitations of the disclosure. The principal features of this disclosure can be employed in various embodiments without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A seed coating composition comprising a co-polymer and bacteria, wherein said seed coating composition increases longevity of the bacteria inoculant on a coated seed.

2. The seed coating composition of claim 1, wherein said co-polymer comprises CPFAPH.

3. The seed coating composition of claim 1, wherein said seed is a glycine max seed.

4. The seed coating composition of claim 1, wherein said seed is a legume.

5. The seed coating composition of claim 4, wherein said legume seed is selected from alfalfa, chickpea, and bean.

6. The seed coating composition of claim 1, wherein said microorganism is a member of the genus Rhizobium.

7. The seed coating composition of claim 1, wherein said microorganism is a plant growth promoting rhizobacteria.

8. A method to increase microorganism longevity on a seed comprising the step of
coating said seed with the seed coating composition of claim 2, in an amount effective to increase said microorganism longevity.

9. The method of claim 8, wherein said microorganism is a member of the genus Rhizobium.

10. The method of claim 8, wherein said microorganism is a plant growth promoting rhizobacteria.

11. The method of claim 8, wherein said seed is a glycine max seed.

12. The method of claim 8, wherein said seed is a legume.

13. The method of claim 8, wherein said legume seed is selected from alfalfa, chickpea, and bean.

14. A method to improve plant yield comprising the step of coating the seed of said plant with a seed coating composition comprising CPFAPH before coating the seed with a plant growth promoting rhizobacteria.

15. The method of claim 14, further comprising the step of adding a pesticide, insecticide and/or a fungicide.
